(19) United States
(12) Patent Application Publication
(10) Pub. No.: US 2007/0100439 A1
(43) Pub. Date: May 3, 2007

(54) Chordae tendinae restraining ring

(75) Inventors: Vincent J. Cangialosi, Beverly, MA (US); Allan Steingisser, Melrose, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/262,583

(22) Filed: Oct. 31, 2005

(51) Int. Cl. A61F 2/24

(52) U.S. Cl. 623/2.11; 623/2.36

(57) ABSTRACT

A ring for surrounding the chordae tendinae of a heart valve, and a system for delivering the ring. The ring gathers the chordae tendinae into a bundle to effectively shorten the chordae tendinae to resolve or reduce valve leaflet prolapse. The body of the ring has an elongated generally linear delivery configuration and a plurality of annular treatment configurations. The body of the ring is releasably carried within a delivery catheter to a treatment location, and a push rod expels the ring from the delivery catheter. Upon being expelled from the delivery catheter, a second end of the ring body will co-axially align with and insert into a first end of the body to form the ring.

### TECHNICAL FIELD

The present invention relates to a medical device. More particularly, the present invention relates to a ring that treats heart valve malfunction by restraining the chordae that are attached to leaflets of a heart valve.

### BACKGROUND OF THE INVENTION

Heart valves, such as the mitral valve, are sometimes damaged by disease or by aging, which can cause problems with the proper function of the valve. Heart valve problems generally take one of two forms: stenosis, in which a valve does not open completely or the opening is too small, resulting in restricted blood flow; or insufficiency or regurgitation, in which blood leaks backward across a valve that should be closed. Valvular insufficiency may result from a dilated valve annulus, because of heart disease. Alternatively, regurgitation may be caused by mitral valve prolapse, which is considered a genetic disorder rather than a conventional disease. Valve replacement may be required in severe cases to restore cardiac function.

Any one or more of the mitral valve structures, i.e., the anterior and posterior leaflets, the chordae, the papillary muscles or the annulus may be compromised genetically, or by damage from disease or injury, causing the mitral valve insufficiency. Mitral valve regurgitation may occur as the result of the leaflets being moved back from each other by the dilated annulus, or by the valve leaflets prolapsing beyond the valve annulus into the atrium. Thus, without correction, the mitral valve insufficiency may lead to disease progression and/or further enlargement and worsening of the insufficiency. In some instances, correction of the regurgitation may not require repair of the valve leaflets themselves, but simply a reduction in the size of the annulus.

A variety of techniques have been attempted to reduce the diameter of the mitral annulus and eliminate or reduce valvular regurgitation in patients with incompetent valves. Current surgery to correct mitral regurgitation in humans includes a number of mitral valve replacement and repair techniques.

Valve replacement can be performed through open-heart surgery, open chest surgery, or percutaneously. The native valve is removed and replaced with a prosthetic valve, or a prosthetic valve is placed over the native valve. The valve replacement may be a mechanical or a biological valve prosthesis. The open chest and percutaneous procedures avoid opening the heart and cardiopulmonary bypass. However, the valve replacement may result in a number of complications including a risk of endocarditis. Additionally, mechanical valve replacement requires subsequent anticoagulation treatment to prevent thromboembolisms.

As an alternative to valve replacement, various surgical valve repair techniques have been used including quadrangular segmental resection of a diseased posterior leaflet; transposition of posterior leaflet chordae to the anterior leaflet; valvuloplasty with plication and direct suturing of the native valve; substitution, reattachment or shortening of chordae tendinae; and annuloplasty in which the effective size of the valve annulus is contracted by attaching a prosthetic annuloplasty ring to the endocardial surface of the heart around the valve annulus. The annuloplasty techniques may be used in conjunction with other repair techniques.

Typically, such rings are sutured along the posterior mitral leaflet adjacent to the mitral annulus in the left atrium. The rings either partially or completely encircle the valve, and may be rigid or flexible/non-elastic. All of these surgical procedures require cardiopulmonary bypass, though some less and minimally invasive techniques for valve repair and replacement are being developed.

Although mitral valve repair and replacement can successfully treat many patients with mitral valve insufficiency, techniques currently in use are attended by significant morbity and mortality. Most valve repair and replacement procedures require a thoractomy, to gain access into the patient's thoracic cavity. Surgical intervention within the heart generally requires isolation of the heart and coronary blood vessels from the remainder of the arterial system and arrest of cardiac function. Open chest techniques with large sternum openings are typically used. Those patients undergoing such techniques often have scarring retraction, tears or fusion of valve leaflets as well as disorders of the subvalvular apparatus.

Recently other surgical procedures have been provided to reduce the mitral annulus using a less invasive surgical technique. According to this method, a prosthesis is transvenously advanced into the coronary sinus and the prosthesis is deployed within the coronary sinus to reduce the diameter of the mitral annulus. This may be accomplished in an open procedure or by percutaneously accessing the venous system by one of the internal jugular, brachial, radial, or femoral veins. The prosthesis is tightened down within the coronary sinus, located adjacent the mitral annulus, to reduce the mitral annulus.

While the coronary sinus implant provides a less invasive treatment alternative, the placement of the prosthesis within the coronary sinus may be problematic for a number of reasons. Sometimes the coronary sinus is not accessible. The coronary sinus on a particular individual may not wrap around the heart far enough to allow enough encircling of the mitral valve. Also, leaving a device in the coronary sinus may result in formation and breaking off of thrombus that may pass into the right atrium, right ventricle and ultimately the lungs causing a pulmonary embolism. Another disadvantage is that the coronary sinus is typically used for placement of a pacing lead, which may be precluded with the placement of the prosthesis in the coronary sinus.

It would be desirable, therefore, to provide a method and device for reducing mitral valve regurgitation that would overcome these and other disadvantages.

### SUMMARY OF THE INVENTION

One aspect of the present invention provides a ring for surrounding the chordae tendinae of a diseased heart valve. The ring effectively shortens the chordae tendinae by altering the direction vector of the chordae tendinae relative to the valve leaflets and thereby changing the vector of forces (including resistive forces) exerted on the valve leaflets by the chordae tendinae. This effective shortening acts to improve valve function. The ring is formed from a hollow tube made from a shape memory material. One end of the tube is flared and the opposite end is sized such that it can fit co-axially inside the flared end.

The ring has a tubular linear delivery configuration. The ring may have one of several annular treatment configurations. The ring is elastically deformable between an annular treatment configuration and the linear delivery configuration. In at least one embodiment, the ring has a shape memory of the annular treatment configuration.

A system of the present invention includes a ring for surrounding the chordae tendinae of a diseased heart valve. The ring is releaseably carried within a delivery catheter, which has a push rod to release the ring from the catheter.

Another aspect of the present invention provides a method for treating a diseased heart valve using the rings disclosed herein. The method comprises delivering a self-forming annular ring made from a hollow tube in a lumen of a catheter proximate the diseased heart valve, releasing the self forming annular ring and encircling chordae tendinae of the diseased heart valve with the ring such that one end of the hollow tube fits co-axially into the opposite end to form the ring.

The foregoing and other features and advantages of the invention will become further apparent from the following detailed description of the presently preferred embodiments, read in conjunction with the accompanying drawings, which are not to scale. The detailed description and drawings are merely illustrative of the invention, rather than limiting the scope of the invention being defined by the appended claims and equivalents thereof.

### BRIEF DESCRIPTION OF THE DRAWINGS

FIG. 1 shows a detailed illustration of one embodiment of a heart valve repair system including a chordae tendinae ring in accordance with the present invention.

FIG. 2 shows one embodiment of a ring of the heart valve repair system in accordance with the present invention.

FIG. 3 shows the ring of the heart valve repair system illustrated in FIG. 2 in a closed configuration in accordance with the present invention.

FIGS. 4 and 5 illustrate the procedure for placement of one embodiment of the ring around the chordae tendinae in accordance with the present invention.

FIGS. 6 and 7 illustrate an alternate procedure for placement embodiment of the ring around the chordae tendinae in accordance with the present invention.

### DETAILED DESCRIPTION

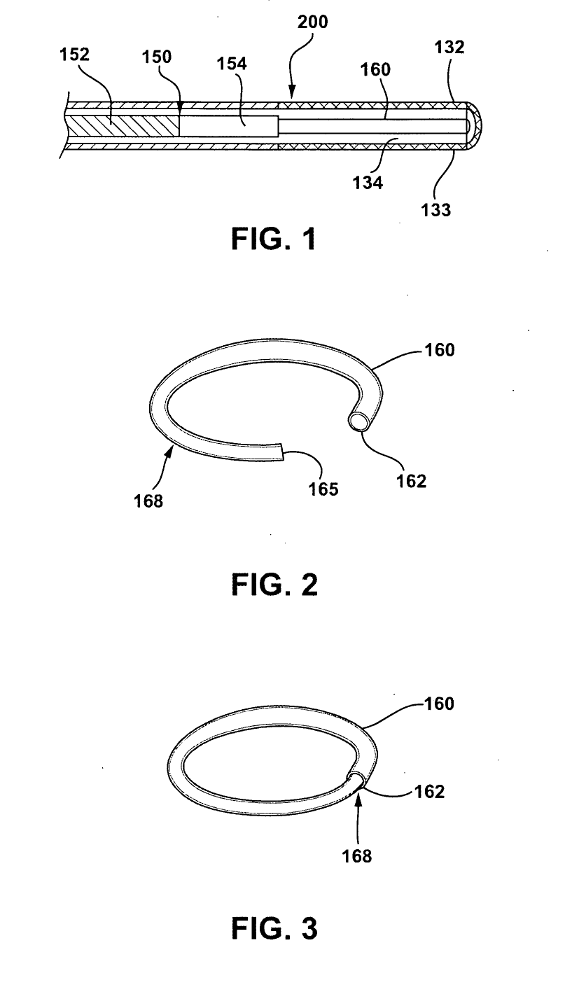

The invention will now be described in detail below by reference to the drawings, wherein like numbers refer to like structures. Referring to FIG. 1, there is shown a detailed illustration of a heart valve repair system 100. Heart valve repair system 200 comprises an elongate delivery device having a delivery catheter 132 and push rod 150. Delivery catheter 132 includes lumen 134 and distal end 133. System 200 further includes ring 160 disposed within lumen 134 of delivery catheter 132. In one embodiment, push rod 150 includes rigid proximal portion 152 and flexible distal portion 154. Flexible portion 154 contacts ring 160. In one embodiment, push rod 150 is moved in an axial direction to push ring 160 from delivery catheter 132.

Elongated push rod 150 may be solid or a hollow rod closed at its distal end for contact with ring device 120. Push rod 150 may be composed of any material that is sufficiently flexible to traverse a tortuous path to the left ventricle, and sufficiently incompressible to controllably push ring 160 out of delivery catheter 132. Examples of suitable plastic materials to fabricate push rod 150 include amides, polyimides, polyolefins, polyesters, urethanes, thermoplastics, thermoset plastics, and blends, laminates or copolymers thereof. Push rod 150 may also be composed of metal, such as a core wire with a coiled spring at the distal end. Push rod 150 may also have a lubricious coating on the outer surface to provide lubrication between the inner surface of delivery catheter 132 and the outer surface of push rod 150.

Delivery catheter 132 may include reinforced portion 135 to help maintain ring 160 in its deformed linear delivery configuration. Reinforced portion 135 may incorporate a braided material or other stiffening member. In another embodiment, reinforced portion 135 may comprise a pre-shaped curve to assist in accurately placing ring 160 within the patient's cardiac anatomy. A thermoplastic material can be used in reinforced portion 135 to form and retain the pre-shaped curve.

Ring 160 is held within delivery catheter 132 in a linear delivery configuration so that it may be delivered via catheter 132 to the chordae tendinae. The linear delivery configuration is obtained by deforming ring 160 from its annular treatment configuration and inserting the linear deformed ring into the delivery catheter 132. Ring 160 can be deformed into the delivery configuration before or during insertion into the delivery catheter 132.

Ring 160 may be composed of a biocompatible material having sufficient elastic properties to permit deformation from the annular treatment configuration into the linear delivery configuration and subsequent re-formation of the device back into the annular treatment configuration. Materials for use in making embodiments of the rings disclosed herein include any suitable biocompatible material that has shape memory properties. Such materials can include shape memory metals, shape memory alloys, and plastics having shape memory properties.

FIGS. 2 and 3 illustrate a device of the current invention having a tubular body that forms a generally circular ring 160 when fully deployed. The tubular body may have a round or other cross-section that would allow formation of a ring as described herein. Ring 160 is composed of material that is formed and set in the closed ring configuration. In one embodiment, the ring may be formed by wrapping the tube around a mandrel or other device suitable for forming the ring. Heat setting the formed ring provides shape memory to the material so that ring 160 will return to the annular treatment configuration from the deformed linear delivery configuration when ring 160 is delivered to more than one chordae tendinae, possibly all of the chordae tendinae.

The ring is formed from a tube having a channel communicating therethrough. The tube made from some shape memory material as described above. A clinician can deliver the ring to a treatment site by using an elongated delivery device (such as the catheter described herein) to a ventricle in an elongated, essentially linear delivery configuration (as depicted in FIG. 1). When the ring is deployed from the delivery device, the shape memory properties of the material from which the ring is constructed, cause it to assume a circular, ring shape. Those familiar with shape memory materials will readily understand that the ring can be formed into the elongated delivery configuration through changes in temperature or through the use of stress.

As can be seen from the figure, a first end 162 of the tubular body that forms the ring 160 is flared and a second end 165 is smaller such that it fits inside the first end 162 in a co-axial manner. In the depicted embodiment the first end is flared for only a portion of the length of the body, but in another preferred embodiment the diameter of the tubular body can be tapered along its entire length so that a narrower end will fit co-axially into a larger end.

When the tubular body is deployed from the delivery device, it begins to form into the ring shape. During the formation of the ring, the body preferably surrounds more than one of the chordae tendinae such that chordae from each of the valve leaflets is surrounded by the ring. Once the tubular body assumes a ring shaped deployment configuration, in which the smaller diameter second end 165 aligns co-axially and inserts into the larger diameter first end 162.

Those with skill in the art will recognize that the lengths and transverse dimensions of ring may be selected to accommodate the size and shape of a specific patient's heart structure. In at least one embodiment, the tubular body has a portion with a diameter that is greater than the diameter of the opening in the first end 162 of the body. When the tubular body is set into the shape of the ring, it is set such that that after the second end is co-axially aligned with the first end it will be inserted to a point 168 where the diameter of the body is larger than the opening in the first end. When the ring is deployed and wraps around the chordae tendinae, it may continue to form the ring shape until the point 168 where diameter of the body prevents further penetration into the first end 162.

In another embodiment, the body of the ring is not completely hollow. Instead, a cavity extends for a distance along the body from an opening in the first end. When the ring transitions to its annular treatment configuration, the second end will extend into the first end until it reaches the end of the cavity.

In one embodiment of the device, the second end penetrates into the first end of the ring until contraction is stopped by the resistive force of the chordae tendinae. In yet another embodiment, the ring does not have some location where the diameter is wider than the opening in the first end that will stop contraction, but it is pre-set to allow the second end to penetrate for a pre-determined distance into the first end. In all of the embodiments of the current invention, the diameter of the finished ring allows the ring to secure the chordae tendinae and aid in holding the valve leaflets closer together to help prevent regurgitation of blood into the left atrium.

It should be noted, that while the FIGS. show embodiments of the invention having a generally circular cross section (taken along the long axis at a right angle to that axis), other cross sections can be appropriate for the devices/rings disclosed herein. Thus, this document should not be read as limiting this invention to generally cylindrical tubes having circular cross-sections. Instead, this document should be read to include non-cylindrical bodies having cross-sections of shapes that would be appropriate for securing the chordae tendinae of a heart valve. Such bodies could have elliptical cross sections, a square with rounded exterior corners, an equilateral triangle with the exterior of the apexes rounded off, and other shapes so long as the tubes were not shaped such that they would cut, nick, or otherwise cause damage to the chordae tendinae due to sharp edges.

It should also be noted that while the embodiments of the device shown in the attached FIGS. are depicted and described in their annular treatment/deployment configurations as rings having a generally circular shape, in other embodiments the rings do not have to form a perfect circle. Thus, the annular treatment/deployment configuration of the device may be a ring shaped in a circle, it may be elliptically shaped, or it may have any shape suitable for capturing at least one chordae tendinae from a plurality of heart valve leaflets and changing the direction vectors of the chordae tendinae to effectively shorten the chordae tendinae as described herein.

Also, in at least one embodiment of the device, the body is not hollow along its entire length. In such an embodiment, the first end of the body has an opening and a cavity therein such that the second end of the device can fit into the in the first end.

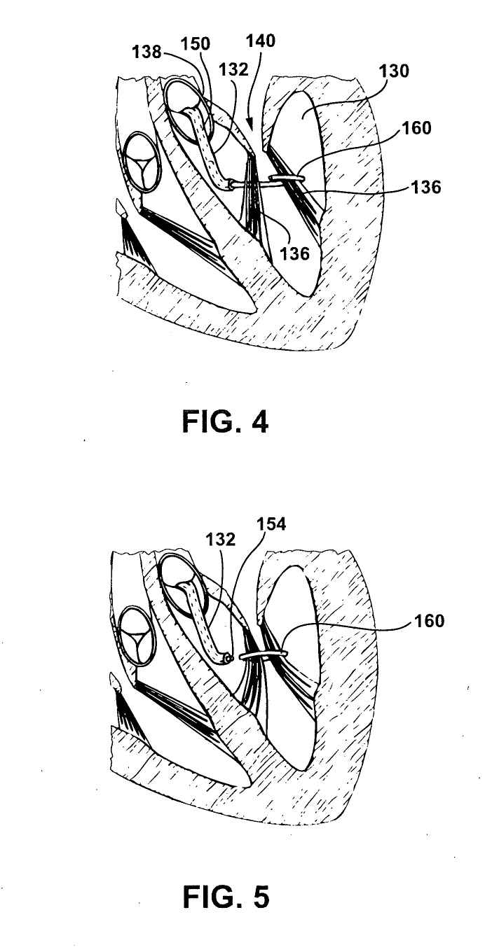

FIGS. 4 and 5 illustrate the deployment of ring 160 into an annular treatment configuration around chordae tendinae 136 of a mitral valve. As illustrated in FIG. 4, delivery catheter 132 has been advanced transluminally through the patient's vasculature and through aortic valve 138 into the left ventricle. Those with skill in the art will recognize that the devices and methods disclosed herein may be applied alternatively to the chordae tendinae within the right ventricle. The FIGS. show an embodiment of a heart valve repair system wherein ring 160 is held in a deformed linear delivery configuration within an elongate delivery element. The collapsible ring can be delivered via a percutaneous transluminal route, using a catheter. Alternatively, the ring can be delivered surgically, using a cannula, a trocar or an endoscope as the elongate delivery element.

For the exemplary case of the heart valve repair system shown in FIGS. 4 and 5 an elongate element is first placed to provide a path from the exterior of the patient to left ventricle 130. In one embodiment, the elongate element is catheter 132. Ring 160 can then be advanced through a delivery lumen so that ring 160 is located at the mitral valve chordae tendinae 136 for deployment. FIG. 4 illustrates an aortic approach to the left ventricle: catheter 132 may be inserted into a femoral artery, through the aorta, through aortic valve 138 and into left ventricle 130. Those skilled in the art will appreciate that alternative paths are available to gain access to the left ventricle. For surgical approaches with an open chest, the elongate delivery element can be a trocar or cannula inserted directly in the aortic arch. The elongate delivery element can then follow the sarne path as in the percutaneous procedure to reach the left ventricle. The left ventricle can also be accessed transluminally through the patient's venous system to the right ventricle, and then using trans-septal techniques to traverse the ventricular septum. Related transluminal or surgical approaches can be used to access the chordae tendinae of the tricuspid valve.

As shown in FIG. 4, delivery catheter 132 is advanced until the distal end is adjacent chordae tendinae 136 of the mitral valve. The advancement of delivery catheter 132 to the chordae tendinae may be monitored by methods known in the art such as fluoroscopy and ultrasonography. In one embodiment, delivery catheter 132 and/or push rod 150 may include radiopaque markers to improve fluoroscopic visualization of the component. To deploy ring 160, push rod 150 is advanced towards the distal end of delivery catheter 132.

As illustrated in FIGS. 4 and 5, the continued advancement of push rod 150 extends more of ring 160 out of catheter 132, and, due to the elastic shape memory of the ring material, ring 160 begins to form around the chordae tendinae. Upon complete deployment, ring 160 surrounds the chordae tendinae. In another technique, ring 160 is deployed to form the annular treatment configuration by holding push rod 150 in position while retracting delivery catheter 132. In this technique, ring 160 will reform into the annular treatment configuration as delivery catheter 132 is withdrawn in a proximal direction.

Once formed, the inner diameter of ring 160 contacts the chordae tendinae. Further, the inner diameter of the ring 160 is sized to draw the chordae tendinae closer together to form a bundle to effectively improve valve function. The ring alters the direction vector of the chordae tendinae relative to the valve leaflets and thereby changing the vector of forces (including resistive forces) exerted on the valve leaflets by the chordae tendinae. This alteration of the vectors is referred to hereinafter as “effective shortening” of the chordae tendinae. This shortening of the chordae tendinae acts to improve valve function and in at least one embodiment, the valve function is improved based on improved leaflet coaption. Further, the placement of the ring simulates surgical techniques such as chordal transposition or papillary muscle repositioning. In some applications, the tension that the ring provides in the chordae tendinae may reduce the diameter of the mitral valve annulus, resulting in more complete closing of the leaflets to eliminate valve regurgitation.

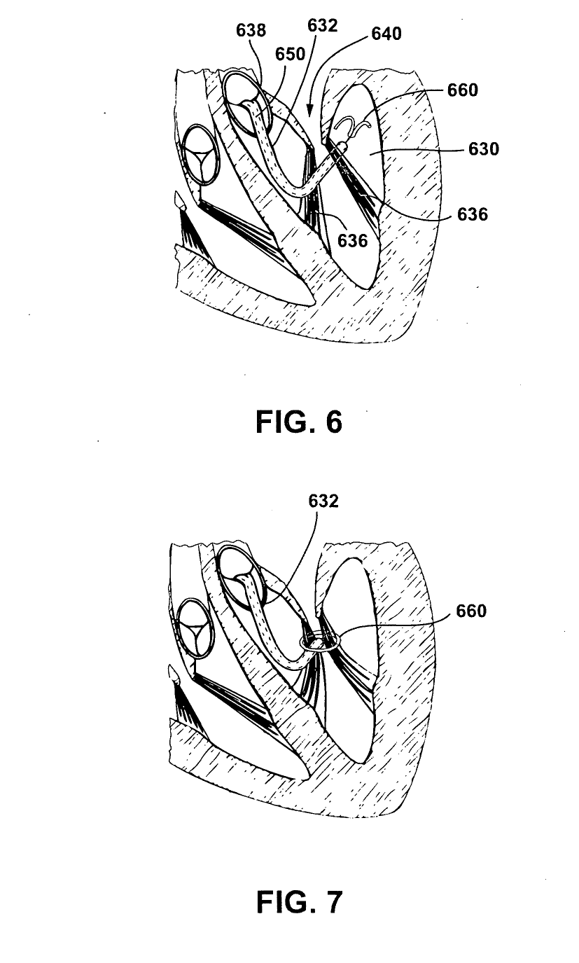

FIGS. 6 and 7 illustrate an alternate procedure for deployment of ring 660 into an annular treatment configuration around chordae tendinae 636 of the mitral valve. As illustrated in the FIGS., delivery catheter 632 has been advanced transluminally through the patient's vasculature and through aortic valve 638 into the left ventricle. Those with skill in the art will recognize that the devices and methods disclosed herein may be applied alternatively to the chordae tendinae within the right ventricle. The FIGS. show an embodiment of a heart valve repair system wherein ring 660 is folded in approximately half and inserted into a delivery device, where it is held in the delivery configuration within the device. The collapsible ring can be delivered via a percutaneous transluminal route, using a catheter taking the routes through the vasculature that are described above. Alternatively, the ring can be delivered surgically, using a cannula, a trocar or an endoscope as the elongate delivery element.

As shown in FIG. 6, the delivery device 632 is advanced until the distal end is adjacent to and between the chordae tendinae 636 of the two mitral valve leaflets. The advancement of delivery catheter 632 to the chordae tendinae may be monitored by methods known in the art such as fluoroscopy and ultrasonography. In one embodiment, delivery catheter 632 and/or push rod may include radiopaque markers to improve fluoroscopic visualization of the component. To deploy ring 660, the push rod is advanced towards the distal end of delivery catheter 632.

As illustrated in FIGS. 6 and 7, the continued advancement of the push rod 650 extends more of ring 660 out of catheter 632, and, due to the elastic shape memory of the ring material, the two ends of the tubular body begin to wrap back around the chordae tendinae and begin to form ring 660 around the chordae tendinae. If the push rod is attached to the ring it can then be detached either by applying tension to the rod or using an additional catheter sheath to push against the ring. In at least one embodiment, the ring is fused to the push rod and electrical current is used to release the fused ring from the rod.

Once formed, the inner diameter of ring 660 contacts the chordae tendinae. Further, the inner diameter of the ring 660 is sized to draw the chordae tendinae closer together to form a bundle to effectively achieve chordal shortening. As noted above, this shortening of the chordae tendinae resolves or reduces valve leaflet prolapse. Further, the placement of the ring simulates surgical techniques such as chordal transposition or papillary muscle repositioning. In some applications, the tension that the ring provides in the chordae tendinae may reduce the diameter of the mitral valve annulus, resulting in more complete closing of the leaflets to eliminate valve regurgitation.

To use the devices of the invention disclosed herein, a clinician begins by delivering a ring proximate the chordae tendinae of the heart valve to be repaired. The ring may be delivered by a delivery catheter as is well known in the art. In one embodiment, the elongate delivery element includes a catheter with a lumen and a push rod positioned within the lumen of the catheter. The ring is held in a deformed linear delivery configuration within the catheter. Once properly positioned, the ring is released from the catheter. The ring may be extended by pushing the ring from the catheter using the pushrod. In another embodiment, the catheter forms a retractable sleeve and the push rod acts as a holding device to hold the ring in a desired position adjacent the chordae tendinae. Then, once positioned properly, the catheter is retracted from the ring allowing the ring to be deployed.

During deployment, the ring surrounds the chordae tendinae of the heart valve by transitioning from the linear delivery configuration to the deployment or annular treatment configuration. Once fully deployed the chordae are completely encircled whereupon, the ring forms a bundle of the chordae tendinae to achieve chordal shortening as described above.

The chordae tendinae restraining rings disclosed herein are used to assist in reducing regurgitation of a heart valve. As described above, the rings can be delivered via catheters through the vasculature of a patient. While most such procedures are envisioned as being performed on the mitral valve, the devices can be used on the other side of the heart as well.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A device for surrounding a plurality of chordae tendinae attached to leaflets of a heart valve comprising:
an elongated, body being constructed from a material having shape memory properties;
the body having an open first end, a second, and a cavity that extends at least partially along the length of the body from the first end;
the first end of the body being larger in size than the second end of the body;
the device having a linear delivery configuration such that the device can be delivered to a location adjacent the chordae tendinae of a heart valve in an elongate delivery device; and
the device having an annular treatment configuration with a shape such that after the device is expelled from the delivery device the second end of the body is co-axially aligned with and inserted into the first end of the body and the device can surround at least two of the chordae tendinae of a heart valve.

2. The device of claim 1 wherein the second end is open and the elongated body is generally hollow such that the cavity that extends at least partially along the length of the body extends along the entire length of the body.

3. The device of claim 1 wherein the shape memory material is a material chosen from a group consisting of: a nitinol alloy, a stainless steel, a cobalt-based alloy, an MP35N® alloy, an Elgiloy® alloy, an engineering plastic, an amide, a polyimide, a polyolefin, a polyester, a urethane, a thermoplastic, a thermoset plastic, and a blend, a laminate and a copolymer of the above materials.

4. The device of claim 1 wherein the elongate delivery device is selected from the group consisting of a catheter, a trocar, a cannula, and an endoscope.

5. The device of claim 1 wherein the annular treatment configuration of the device has a shape selected from a group consisting of: a ring and an ellipse.

6. The device of claim 1 wherein the body has a cross-sectional shape along its length that is selected from the group consisting of circular, elliptical, square, and triangular.

7. The device of claim 1 wherein when the device is in use it will assume the annular treatment configuration such that the second end of the body is co-axially aligned with and inserted into the first end of the body and the device surrounds and secures at least one chordae tendinae from each leaflet of a heart valve such that the direction vectors of the chordae tendinae are altered by the device.

8. The device of claim 1 wherein the elongate body has a shape memory of the annular treatment configuration to which the body tends to reform after having been deformed to the linear delivery configuration.

9. The device of claim 1 wherein the body is folded in half when the body is in a linear delivery configuration inside of an elongate delivery device.

10. A device for surrounding a plurality of chordae tendinae attached to leaflets of a heart valve comprising:
an elongated, generally hollow body being constructed from a material having shape memory properties;
the body having an open first end, an open second end, and a channel communicating therethrough;
the first end of the body being larger in diameter than the second end of the body;
the device having a linear delivery configuration such that the device can be delivered to a location adjacent the chordae tendinae of a heart valve in an elongate delivery device; and
the device having an annular treatment configuration with a shape such that after the device is expelled from the delivery device the second end of the body is co-axially aligned with and inserted into the first end of the body and the device can surround at least two of the chordae tendinae of a heart valve.

11. The device of claim 10 wherein the shape memory material is a material chosen from a group consisting of: a nitinol alloy, a stainless steel, a cobalt-based alloy, an MP35N® alloy, an Elgiloy® alloy, an engineering plastic, an amide, a polyimide, a polyolefin, a polyester, a urethane, a thermoplastic, a thermoset plastic, and a blend, a laminate and a copolymer of the above materials.

12. The device of claim 10 wherein the elongate delivery device is selected from the group consisting of a catheter, a trocar, a cannula, and an endoscope.

13. The device of claim 10 wherein the annular treatment configuration of the device has a shape selected from a group consisting of: a ring and an ellipse.

14. The device of claim 10 wherein the body has a cross-sectional shape along its length that is selected from the group consisting of circular and elliptical.

15. The device of claim 10 wherein when the device is in use it will assume the annular treatment configuration such that the second end of the body is co-axially aligned with and inserted into the first end of the body and the device surrounds and secures at least one chordae tendinae from each leaflet of a heart valve such that the valve leaflets are closer together than they were before the device encircled the chordae tendinae.

16. The device of claim 10 wherein the elongate body has a shape memory of the annular treatment configuration to which the body tends to reform after having been deformed to the linear delivery configuration.

17. The device of claim 10 wherein the body is folded in half when the body is in a linear delivery configuration inside of an elongate delivery device.

18. A system for treating a heart valve by surrounding a plurality of the chordae tendinae attached to the valve leaflets comprising
an elongate delivery catheter having a lumen; and
a treatment device having an elongated, generally hollow body being constructed from a material having shape memory properties;
the body having an open first end, an open second end, and a channel communicating therethrough;
the first end of the body being larger in diameter than the second end of the body;
the device having a linear delivery configuration such that the device can be releasably disposed in the delivery catheter; and
the device having an annular treatment configuration with a shape such that after the device is expelled from the delivery device the second end of the body is co-axially aligned with and inserted into the first end of the body and the device can surround at least two of the chordae tendinae of a heart valve.

19. The system of claim 18 further comprising a push rod slidably disposed within the lumen of the delivery catheter and being capable of pushing the device out of the delivery catheter.

20. The system of claim 18 wherein the elongate body of the treatment device has a shape memory of the annular treatment configuration to which the body tends to reform after having been deformed to the linear delivery configuration.
